(19) United States
(12) Patent Application Publication
(10) Pub. No.: US 2018/0200191 A1
(43) Pub. Date: Jul. 19, 2018

(54) Multi Component Pill and Method(s) of Use

(71) Applicant: Theresa M. Lay, Denver, CO (US)

(72) Inventor: Theresa M. Lay, Denver, CO (US)

(21) Appl. No.: 15/874,651

(22) Filed: Jan. 18, 2018

Related U.S. Application Data
(60) Provisional application No. 62/448,113, filed on Jan. 19, 2017.

(51) Int. Cl. A61K 9/20; A61K 9/48

(52) U.S. Cl. CPC A61K 9/2072; A61K 9/48

(57) ABSTRACT

A multi component pill and method(s) of use are described. The multi component pill comprises disparate nutrient or vitamin components in a unitary pill. Typically, the pill comprises at least first and second components with one component surrounding at least a portion of the other component. Variations can include a third component affixed to the first and second component. The pill may either be swallowed whole and in-tact, or in other variations, the second and/or third component may be bitten or broken off from the first core component and then chewed and swallowed prior to ingestion of the first inner core component.

### CROSS-REFERENCE TO RELATED APPLICATION

The present application claims priority to and incorporates by reference U.S. provisional Patent Application No. 62/448,113, filed Feb. 19, 2017, having the same inventor and title as the present application.

### BACKGROUND

Vitamin/daily health packs are commonly used to supply daily supplement needs in one convenient package. They typically include anywhere from two to six capsules or pills that are to be taken individually by a user. The pills are provided separately for a variety of reasons, but one significant reason is that when combined, certain nutritional components may not be compatible with others. Stated another way, the nutritional value offered by particular nutrients may be compromised or reduced if directly exposed to another nutrient. In some instances, the absorption of certain vitamins and/or minerals can be enhanced in the presence of other vitamins or minerals, though it may not be desirable to combine particular minerals and/or vitamins together in one pill prior to ingestion. Further, liquid components often necessitate separate pills or capsules from that of their dry counterparts to maintain the integrity of the vitamin or nutrient. While providing certain minerals and/or vitamins in separate capsules or pills can address these concerns, users often find the task of taking upwards of six individual capsules daily to be burdensome and inefficient.

### BRIEF DESCRIPTION OF THE DRAWINGS

FIG. 1 is a perspective view of a pill with a torus-shaped second component included according to an embodiment.

FIG. 2 is a cross sectional end view of a pill with a torus-shaped second component included according to an embodiment.

FIG. 3 is a perspective view of a pill with outer shell of one composition and with inner core of another composition according to an embodiment.

FIG. 4 is a cross sectional end view of a pill with outer shell of one composition and with inner core of another composition according to an embodiment.

FIG. 5 is a cross sectional end view of a pill with a third component layer between the inner core and outer shell according to an embodiment.

FIG. 6 is a perspective view of a pill with a capped end according to an embodiment.

FIG. 7 is a perspective view of a pill with a circumferential groove provided on its outer shell according to an embodiment.

FIG. 8 is a perspective view of a pill with detached outer shell comprising two disparate compositions according to an embodiment.

FIG. 9 is a perspective view of a pill with detachable outer shell comprising two disparate compositions according to an embodiment.

### DETAILED DESCRIPTION

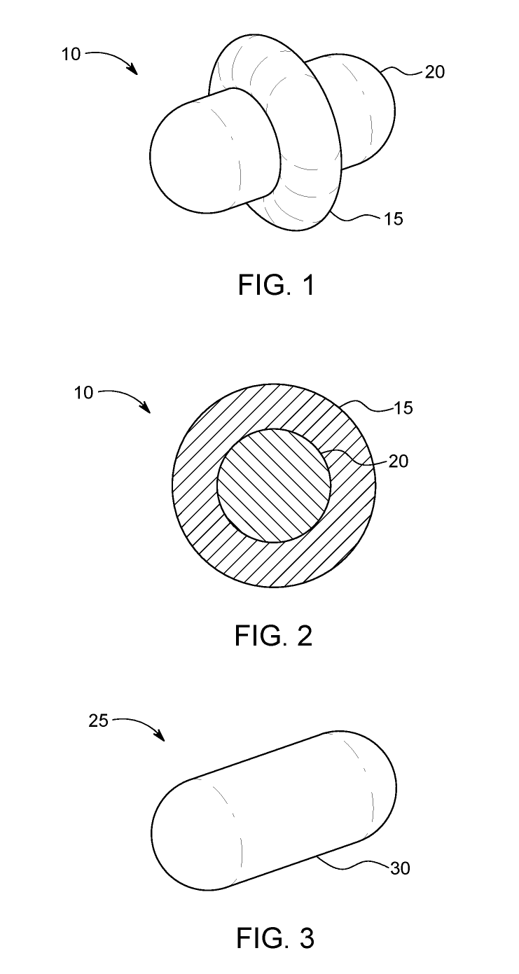

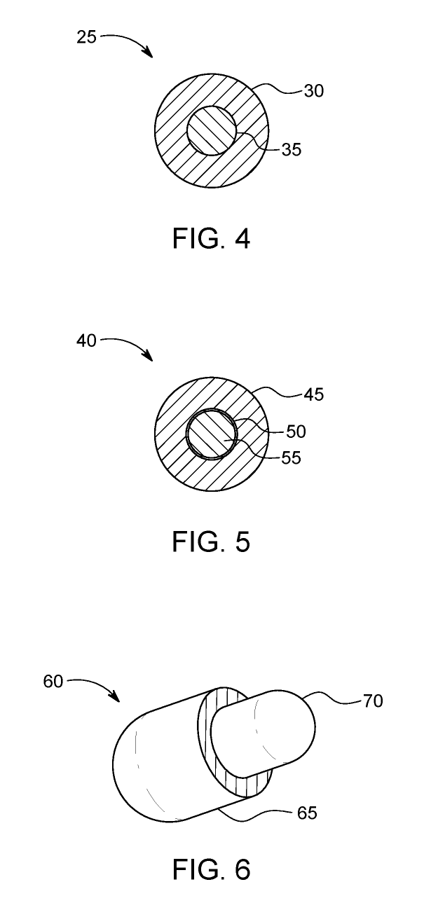

Embodiments of the present invention include a multi component pill, also referred to as a two-component pill or pill. Various embodiments of the pill are illustrated in FIGS. 1-9 and described herein. As can be appreciated and is also discussed herein, other variations are also possible as would be obvious to someone of ordinary skill given the benefit of this disclosure.

Embodiments of the multi component pill are designed and configured wherein components comprising disparate nutritional or active ingredients can be provided together in a singular compact and portable digestible form. Embodiments typically comprise an oblong shaped body comprising digestible nutrient or medicinal content and a partially or fully enclosed second component comprising a disparate digestible nutrient or medicinal content. Variations can also include a third component comprising distinct digestible nutrient or medicinal content.

In one embodiment, a two-component pill comprises an oblong shaped first component encompassed by a torus-shaped second component. The second component comprises a tablet which can be separately pressed into a torus-shaped form, and then slid over the first component and held in place by friction and/or a digestible adhesive material. Alternatively, the second component can be molded over the first component. The second component tablet may comprise a chewable mixture, that is either bitten or broken off the first component and chewed and swallowed prior to ingesting the first component. The first component pill can be of the type that is intended to be swallowed in whole without chewing, such as a capsule or caplet, or of the type that is intended to be chewable, such as a tablet, allowing for the entire pill to be chewable.

In another embodiment, a two-component pill comprises an oblong shaped inner first component fully enclosed within a second, oblong shaped outer component. In one variation, a v-shaped circular circumferential groove is formed on the outer second component, either during molding or in a subsequent operation, to permit the outer second component to be split apart and removed from the inner first component. This variation would be most likely utilized wherein the outer second component is intended to be chewable, such as a tablet, and the inner first component is intended to be swallowed in whole without chewing, such as a capsule or caplet. Once the second component has been either bitten or broken off the first component and chewed and swallowed, the second component may be ingested. In another variation, a third component thin barrier layer is provided between the first and second component. The third component layer can comprise medicinal or nutritional ingredients, and/or may provide a protective barrier between the first and second component wherein the first component is prevented contact from the second component.

In another embodiment, a second component comprises a cup-shaped form and is encased over a portion of the body of the first component. In this embodiment, the second component can be formed separately from the first component, with the portion of the first component that is to be encased being inserted into the second component. Alternatively, the second component can be molded over a portion of the first component. In this embodiment, the outer second component is intended to be chewable, such as a tablet. As in other embodiments, the first component pill can be of the type that is intended to be swallowed in whole without chewing, such as a capsule or caplet, or of the type that is intended to be chewable, such as a tablet, allowing for the entire pill to be chewable.

In another embodiment, the multi component pill is described as having three separate and distinct components. In one variation, the first component has an oblong shape with generally hemispherical ends, and the second and third components comprise cup shaped elements that fit over the first component and completely or substantially enclose the first component once they are joined together, and may be held in place by friction and/or a digestible adhesive material. This pill may either be swallowed whole and in-tact, or alternatively, the second and third components can be of the type that are intended to be chewable, such as a tablet as discussed in other embodiments, and may be bitten or broken off from the first component and chewed and swallowed prior to ingestion of the first component. As in other embodiments, the first component pill can be the type that is intended to be swallowed in whole without chewing, such as a capsule or caplet, or of the type that is intended to be chewable, such as a tablet, allowing for the entire pill to be chewable.

The individual components comprising the two-component or multi component pill can be fabricated by any number of suitable processes. For instance, where the component comprises a chewable tablet, the tablet can be manufactured by means of any suitable tablet pressing process, such as, but not limited to, powder compaction. During the tablet pressing process, it is important to ensure the correct amount of the active ingredient is present in each tablet. To accomplish this, the ingredients must be well-mixed. Vitamin and/or mineral powders that can become sufficiently homogenous with a simple blending process can be compressed into tablets without further processing prior to compression. Other vitamin and/or mineral ingredients can require a granulation process, such as wet granulation or dry granulation, prior to compression.

In embodiments where the first and second component of the pill comprise a chewable tablet, producing the first and second tablet components separately can allow for different vitamin and/or mineral ingredients having incompatible processing requirements to be subsequently joined in a singular structure without adverse effect to the nutritional value of the individual components. For example, some vitamin and/or mineral ingredients may be sensitive to moisture and heat, while other vitamin and/or mineral ingredients may necessitate wet granulation to become sufficiently well-mixed prior to compression.

Where the component pill is of the type intended to be swallowed in whole, the first component can comprise a capsule, such as a soft-shelled or hard-shelled capsule, or caplet. The first component capsule or caplet can be manufactured by means of any suitable process. For instance, where a component comprises a soft-shelled capsule, the capsule can be produced using the well-known rotary die encapsulation process, wherein a liquid of compatible composition is injected into the capsule as it is formed. Where a component comprises a hard-shelled capsule, the capsule can be produced by filling a smaller-diameter body with the desired vitamin and/or mineral ingredient(s) and then fitting a larger-diameter cap over the filled body to seal it. Where a component comprises a caplet, a tablet is first produced my means of a suitable pressing process and then coated by using either a coating pan or automatic coater.

A soft-shelled capsule, or soft gelatin, typically comprises a single-piece gel encapsulation containing liquid ingredients, such as oils or active ingredients dissolved in oils. Soft gelatins can be ideal for poorly soluble active ingredients, as the liquid fill can contain other ingredients which increase solubility, or for liquid ingredients that are ill-suited for other dosage forms, such as a tablet.

A hard-shelled capsule, or hard capsule, typically comprises a two-piece gel encapsulation containing dry ingredients such as powders or miniature pellets. Hard capsules can be ideal where the compression forces applied during tablet pressing may degrade ingredients, or for certain herbal formulas that may contain plant leaves or roots which can be more easily filled into a hard capsule. Additionally, some users may have difficulty swallowing a pill or experience an upset stomach upon ingestion of certain ingredients. In such instances, the cap of the hard capsule can be easily removed from its body and the contents of the capsule emptied and mixed into a food, such as applesauce. Similarly, the same process can also allow users to limit their dosage where over consumption of certain nutrients or vitamins, such as vitamin C and some B vitamins, may have adverse health effects.

A caplet is a tablet that has been coated after being pressed. Coatings may be used for tablets having unpalatable ingredients or to extend the shelf-life of components that are sensitive to moisture or oxidation. Coatings may also be used to control the rate of dissolution to allow for optimal absorption. For example, where the active ingredient is better absorbed in the stomach, a quick dissolving coating can be used. Where the active ingredient is better absorbed in the large intestine, a coating resistant to stomach-acid can be used to ensure the tablet reaches such point prior to dissolution.

An embodiment comprising a first component capsule or caplet and a second component tablet, capsule or caplet allows for distinct active ingredients, of which their optimal manufacturing method and/or dosage form may differ, to be manufactured individually and subsequently joined together in a singular structure without adverse effect to the integrity of the nutritional or medicinal ingredients of the individual components.

Once formed, the multi component pills can be packaged by any suitable means. They can be bulk packaged in a singular container or they can be individually packed, such as in a flexible cellophane wrapper. Alternatively, they can also be packaged in a blister pack of which the pack may be constructed to prevent damage to the pill by providing crush resistance.

As can be appreciated from the disclosures herein, a variety of manners of joining the various components together in a single convenient unit are contemplated and are intended to include other embodiments and variations that would be of ordinary skill to someone in the art to which the invention pertains given the benefit of this disclosure.

### Terminology

The terms and phrases as indicated in quotation marks (“ ”) in this section are intended to have the meaning ascribed to them in this Terminology section applied to them throughout this document, including in the claims, unless clearly indicated otherwise in context. Further, as applicable, the stated definitions are to apply, regardless of the word or phrase's case, to the singular and plural variations of the defined word or phrase.

The term “or” as used in this specification and the appended claims is not meant to be exclusive; rather the term is inclusive, meaning either or both.

References in the specification to “one embodiment”, “an embodiment”, “another embodiment, “a preferred embodiment”, “an alternative embodiment”, “one variation”, “a variation” and similar phrases mean that a particular feature, structure, or characteristic described in connection with the embodiment or variation, is included in at least an embodiment or variation of the invention. The phrase “in one embodiment”, “in one variation” or similar phrases, as used in various places in the specification, are not necessarily meant to refer to the same embodiment or the same variation.

The term “approximately,” as used in this specification and appended claims, refers to plus or minus 10% of the value given.

The term “about,” as used in this specification and appended claims, refers to plus or minus 20% of the value given.

The terms “generally” and “substantially,” as used in this specification and appended claims, mean mostly, or for the most part.

Directional and/or relationary terms such as, but not limited to, left, right, nadir, apex, top, bottom, vertical, horizontal, back, front and lateral are relative to each other and are dependent on the specific orientation of an applicable element or article, and are used accordingly to aid in the description of the various embodiments and are not necessarily intended to be construed as limiting.

The term “therapeutic,” as used in this specification and appended claims, refers to a component with an intended nutritional or medicinal benefit.

The term “soft-shelled capsule” and “soft gelatin,” as used in this specification and appended claims, refers to a single-piece encapsulation comprising a gelatin or vegetable capsule containing liquid ingredients.

The term “hard-shelled capsule” or “hard capsule,” as used in this specification and appended claims, refers to a two-piece gel encapsulation containing dry ingredients such as but not limited to powders or miniature pellets.

The term “caplet,” as used in this specification and appended claims, refers to a pressed tablet that has been coated.

### A First Embodiment Multi Component Pill

A first embodiment multi component pill 10 is illustrated in FIGS. 1 & 2. The first embodiment multi component pill 10 comprises a pill including a first component 20 produced with an oblong shape having generally hemispherical ends with a torus-shaped second component 15 encircling the first component 20 proximate its longitudinal midpoint. The second component 15 can be frictionally received over the first component 20 or alternatively the first component 15 can be adhesively secured in place with a digestible glue.

In one variation, the first component 20 comprises one of solid supplementary minerals and/or vitamins, and the second component 15 comprises the other of solid supplementary minerals and/or vitamins. In other variations, the first component 20 may be a liquid, such as but not limited to fish or omega 3 oil, contained in a capsule with the second component 15 comprising a solid supplementary mineral and/or vitamin. In yet another variation, the first component 20 may be a combination of vitamins and/or minerals that are compatible and the second component 15 may be another combination of vitamins and/or minerals, which are in whole or part not compatible with the contents of the first component 20.

Where the first component 20 and/or second component 15 comprise a solid supplementary vitamin and/or mineral, the first component 20 and/or second component 15 can comprise a chewable dosage form, such as a tablet, or a dosage form which is intended to be swallowed, such as a capsule or caplet. In one variation, the first component 20 may be a capsule or caplet, and the second component 15 may be a chewable tablet. In another variation, the first component 20 and second component 15 may be a chewable tablet, wherein the entirety of pill 10 comprises chewable components.

Where the first component 20 and/or second component 15 comprises a chewable tablet, the first component 20 and/or second component 15 tablet can be manufactured by means of any suitable tablet pressing process, such as but not limited to powder compaction. Prior to compaction, either the first component 20 and/or second component 15 may be mixed with a simple blending process or may require a wet granulation or dry granulation process to become sufficiently homogenous. In some variations, the composition of the first component 20 may necessitate one method, such as wet granulation, and the composition of the second component 15 may necessitate a different method, such as dry granulation.

Where the first component 20 comprises a hard-shelled capsule, the first component 20 capsule can be produced by filling a smaller-diameter body with the desired vitamin and/or mineral ingredient(s) and then fitting a larger-diameter cap over the filled body to seal it. Where a first component 20 comprises a caplet, a tablet comprising supplementary mineral and/or vitamins is first produced my means of a suitable pressing process, and then coated by using either a coating pan or automatic coater. The coating selected for first component 20 will depend on the composition and desired dissolution rate of the supplementary mineral and/or vitamin.

Where the first component 20 comprises a liquid, the first component 20 can comprise soft-shelled capsule. The first component 20 soft-shelled capsule can be produced using the rotary die encapsulation process, wherein a liquid of compatible composition is injected into the capsule as it is formed.

### A Second Embodiment Multi Component Pill

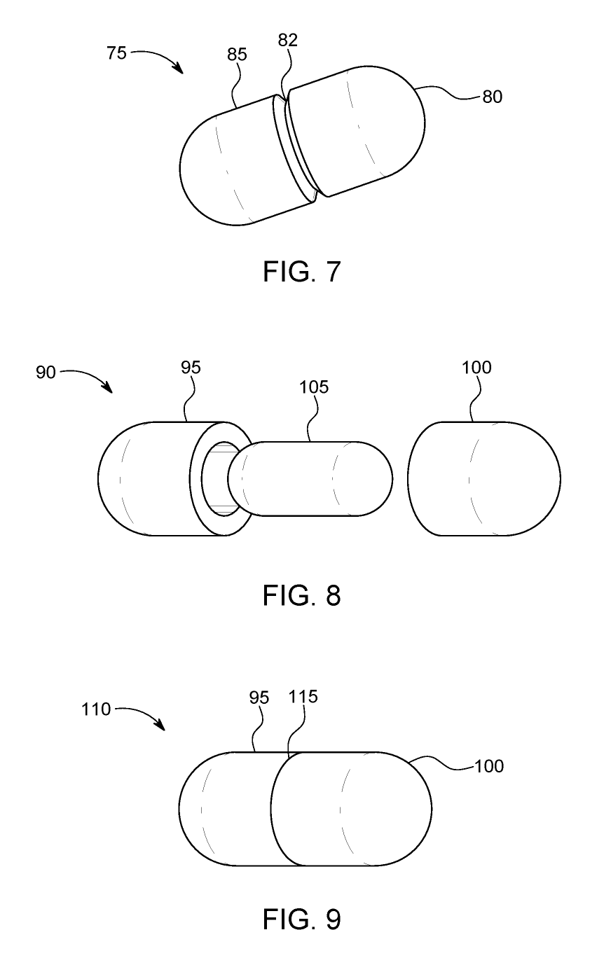

A second embodiment multi component pill 25 is illustrated in FIGS. 3 & 4. The second embodiment multi component pill 25 comprises a pill including first component inner core 35 with an oblong second component 30 fully enclosing the first component 35. The first component 35 may be oblong in shape or it may be any other suitable shape that can be molded over by the second component 30. In one variation, illustrated in FIG. 7, a multi component pill 75 comprises a stress raiser 82, such as a v-shaped circumferential groove, which encircles the second component 80 proximate its longitudinal midpoint.

In one variation, illustrated in FIG. 5, a multi component pill 40 comprises an inner core first component 55 and outer second component 45, and a third component 50 wherein the third component 50 provides a barrier layer between the first component 55 and second component 45. The third component 50 encloses the inner core first component 55 and the oblong-shaped outer second component 45 is formed over the third component 50, fully enclosing both the third component 50 and the first component 55.

The composition and manufacturing of the various components can be similar to the compositions of the components discussed above with reference to FIGS. 1 & 2.

### A Third Embodiment Multi Component Pill

A third embodiment multi component pill 60 is illustrated in FIG. 6. The third embodiment multi component pill 60 comprises a pill including an oblong-shaped inner core first component 70 having generally hemispherical ends and a cap-shaped second component 65 wherein the first component 70 is partially encompassed by the second component 65. The cap-shaped second component 65 can be placed over either of the hemispherical ends of first component 70, and can be frictionally received over the first component 70 or alternatively the first component 70 can be inserted into the second component 70 and adhesively secured in place with a digestible glue.

The composition and manufacturing of the various components can be similar to the compositions of the components discussed above with reference to FIGS. 1 & 2.

### A Fourth Embodiment Multi Component Pill

A fourth embodiment multi component pill 90 is illustrated in FIG. 8. The fourth embodiment multi component pill 90 comprises a pill including first component inner core 105 and enclosing second component 95 and enclosing third component 100. The first component 105 may be oblong in shape or it may be any other suitable shape that can be molded over by the second component 95 and third component 100. The second component 95 and third component 100 each comprise a cup shaped element that form an oblong shaped multi component pill 110 when conjoined, as illustrated in FIG. 9, wherein the second component 95 and third component 100 meet to form a seam 115 proximate the longitudinal midpoint of first component 105.

The second component 95 and third component 100 can be frictionally received over the first component 105 or they can be adhesively secured in place with a digestible glue at seam 115. The composition and manufacturing of the various components can be similar to the compositions of the components discussed above with reference to FIGS. 1 & 2.

### Method(s) of Using a Multi Component Pill

The method of using a multi component pill according to the present invention will vary and is dependent upon the specific construction of the pill and the nature of its components. Some embodiments or variations, such as the embodiments as shown in FIGS. 3, 4, 5, 7, 8 and 9, may be swallowed whole as would a typical vitamin pill or capsule.

In other embodiments, one or more of the components can be chewable. For instance, the second component of the pills in FIGS. 1, 2, 6 and 7 can be bitten or broken off the first component so that it can be chewed and swallowed prior to consumption of the first component, which may then be swallowed whole. In other variations, the first component may also be chewable. In FIG. 7, the outer second component of the pill includes a stress raiser which permits the second component to be broken and separated from the first component and chewed and swallowed prior to consuming the first component.

In one version of the multi component pill, one component is vitamin C and the other component is iron. The vitamin C acts to enhance the absorption of the iron during digestion. In another variation, one component may be calcium and the second component may be vitamin D, which is required for proper calcium absorption in the body. In another variation, one component may be a liquid, such as fish oil, and another component may be a multi-vitamin, as such components may not be shelf-stable when combined into one singular component. In yet another variation, one component may be vitamin A and the other component may be vitamin E, as absorption of one or both may be enhanced in the presence of the other. In another variation, one component may be a probiotic supplement and the second component may comprise a soluble fiber component, as a probiotic taken with soluble fiber may increase the overall effectiveness of the probiotic. In another variation, one component may comprise a quick dissolving pre-workout or energy supplement, while another component may comprise a slow-dissolving recovery supplement, where faster absorption of the pre-workout or energy supplement is desired over that of the recovery supplement. In yet another variation, one of the components may comprise of pharmaceutical drug or medicine, while another component may comprise of another pharmaceutical drug or medicine or alternatively, a nutritional supplement which may either enhance the effect of the pharmaceutical drug or medicine or mitigate its undesirable side-effects.

### Alternative Embodiments and Variations

The various embodiments and variations thereof, illustrated in the accompanying Figures and/or described above, are merely exemplary and not meant to limit the scope of the invention. It is to be appreciated that numerous other variations of the invention have been contemplated, as would be obvious to one of ordinary skill in the art, given the benefit of this disclosure. All variations of the invention that read upon appended claims are intended and contemplated to be within the scope of the invention.

What is claimed is:

1. A unitary pill comprising:
at least a first therapeutic component; and
a second therapeutic component distinct and separate from the first component and at least partially surrounding the first component.

2. The unitary pill of claim 1 further comprising:
a thin barrier layer separating the first therapeutic component and second therapeutic component.

3. The unitary pill of claim 1 further comprising:
a third therapeutic component, the third therapeutic component at least partially surrounding the first therapeutic component.

4. The unitary pill of claim 1 wherein:
the first and second component are chemically incompatible with each other when mixed together prior to ingestion.

5. The unitary pill of claim 4 wherein:
a thin barrier layer separates the first therapeutic component and second therapeutic component.

6. The unitary pill of claim 1 wherein:
the first therapeutic component comprises two or more compatible ingredients.

7. The unitary pill of claim 6 wherein:
the second therapeutic component comprises two or more compatible ingredients.

8. The unitary pill of claim 3 wherein:
the third therapeutic component comprises two or more compatible ingredients.

9. The unitary pill of claim 1 wherein:
the first therapeutic component is in the form of a capsule; and
the second therapeutic component is in the form of a tablet.

10. The unitary pill of claim 1 wherein:
the first therapeutic component is in the form of a tablet; and
the second therapeutic component is in the form of a tablet.

11. The unitary pill of claim 1 wherein:
the first therapeutic component is in the form of a capsule; and
the second therapeutic component is in the form of a caplet.

12. The unitary pill of claim 1 wherein:
the first therapeutic component is in the form of a tablet; and
the second therapeutic component is in the form of a caplet.

13. The unitary pill of claim 1 wherein:
the first therapeutic component is in the form of a caplet; and
the second therapeutic component is in the form of a caplet.

14. The unitary pill of claim 3 wherein:
the first therapeutic component is in the form of a tablet;
the second therapeutic component is in the form of a tablet; and
the third therapeutic component is in the form of a tablet.

15. The unitary pill of claim 3 wherein:
the first therapeutic component is in the form of a capsule;
the second therapeutic component is in the form of a tablet; and
the third therapeutic component is in the form of a tablet.

16. The unitary pill of claim 1 wherein:
the first therapeutic component comprises a liquid contained in a soft-shelled capsule; and
the second therapeutic component comprises a solid.

17. The unitary pill of claim 1 wherein:
the second therapeutic component is flavored and chewable.

18. The unitary pill of claim 1 wherein:
the second therapeutic component substantially surrounds the first component; and
the stress raiser is provided to facilitate the breaking and removal of the second therapeutic component from the first.

19. A method of consuming the unitary pill of claim 1 wherein:
removing the second therapeutic component from the first therapeutic component;
ingesting the first therapeutic component; and
ingesting the second therapeutic component.

20. A method of consuming the unitary pill of claim 18 wherein:
breaking the second therapeutic component at the stress raiser;
removing the second therapeutic component from the first therapeutic component;
ingesting the first therapeutic component; and
chewing the second therapeutic component.
